(19) United States
(12) Patent Application Publication
(10) Pub. No.: US 2018/0093021 A1
(43) Pub. Date: Apr. 5, 2018

(54) ENDOSCOPE HANDLE WITH A RIGID SURGICAL SUCTION FOR ENDOSCOPIC MICRO-NEUROSURGERY OF THE BRAIN

(71) Applicant: SANDEEP SOOD, TROY, MI (US)

(72) Inventor: SANDEEP SOOD, TROY, MI (US)

(21) Appl. No.: 15/283,406

(22) Filed: Oct. 2, 2016

(51) Int. Cl. A61M 1/00; A61B 17/00; A61B 1/015

(52) U.S. Cl. CPC A61M 1/0039; A61B 17/00234; A61B 2217/007; A61B 1/015; A61B 2217/005; A61M 1/0031

(57) ABSTRACT

A handle for endoscope for two handed micro neurosurgery of the brain comprising adjustable rigid surgical suction, Channel for endoscope, Surgical suction control hole.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of endoscopic brain surgery and more specifically to an endoscope handle with rigid surgical suction for two handed dissection surgery. The invention is designed to improve micro-neurosurgery of the brain.

Surgery of the brain is done with a microscope that provides visualization and magnification. The microscope is generally fixed in one position and to move to the area of interest the surgeon physically moves the microscope head holding the handles provided for this purpose. Once the surgeon is satisfied with the position of the microscope, he generally performs the surgery looking through the microscope at the tissue using one instrument in one hand and the other instrument in the other hand. Generally, a surgical suction with low pressure is used in the left hand to keep the field clear of blood. A second instrument is used in the right hand. This may be a dissecting instrument, cutting instrument or any of the myriad of instruments that are used to manipulate tissue as deemed necessary by the surgeon. The two instruments, surgical suction in the left hand and the instrument in the right hand are used in conjunction with each other. The surgical suction in-addition to keeping the field clear of blood is also used to provide counter pressure on the tissue to aid dissection with the second instrument in the right hand. This type of two handed or bimanual ability to manipulate the tissue with two instruments is critical to the objective of surgery which is to dissect and separate abnormal tissue from normal tissue. Bleeding during surgery is controlled with an instrument called bipolar cautery forceps, which is used when needed by replacing the instrument in right hand. During the course of surgery, the surgeon may need to move the position of the microscope head numerous times to get optimal visualization. He may accomplish this by handing instrument in the right hand to the assistant and using the handle on the microscope to move it to a new position and then resume the surgery once satisfied with the new position of the microscope. Small retractor blades may be used to push tissue to one side so as to provide a path through which surgery can be done in the deeper part of the brain.

Some surgeries of the brain may be accomplished using a minimally invasive surgical approach. In these procedures the visualization and magnification is provided by an endoscope. Since the endoscope is much smaller, the incision required for the operation is also smaller, hence called minimally invasive. Endoscope also has an advantage to provide better illumination and magnification compared to a microscope. Surgery is done with an endoscope in two ways. In the first way, through a small incision, the endoscope is introduced into the brain where surgery is to be performed. In the prior art, the endoscope is generally fixed to a holder. An instrument is passed through a co-axial instrument channel present in the endoscope shaft. Generally, the instrument is a flexible jaw that can be opened and closed or scissors that can be used for cutting. However, only one instrument can be introduced at one time. Manipulation with the instrument is limited to pulling or cutting since the instrument can be moved only in and out and not sideways to allow for dissection. Sometimes the surgeon may hold the endoscope in the left hand and introduce the instrument into the endoscope channel through the right hand to accomplish surgery. Since there is only one instrument in the surgical field bimanual or two handed fine dissection is not possible.

In the second way of using the endoscope to accomplish brain surgery, a large port about 1-1.5 cm diameter is passed through the brain to provide a path for operation. This is similar to using small retractor blades to provide a path to the area of interest when surgery is accomplished with a microscope. The endoscope is then fixed to the holder and passed through the port to the area of interest. Through the rest of the space in the port, two instruments are passed, one held in the left hand and the other in the right hand and tissue dissection is accomplished in a two handed bimanual fashion.

The prior art of endoscopic surgery on the brain as described above is plagued with major problems.

Endoscopic brain surgery in the first way has major problems. First, there is only one instrument channel and the instruments can be moved only in one axis, i.e. in and out. Tissue dissection to accomplish surgery in this manner is significantly more difficult and time consuming. Second, the position of the endoscope is fixed by the holder. As a result, to move to a different area of interest the surgeon has to stop, release the endoscope from the holder and reposition the endoscope. This is not only frustrating but also tiring and time consuming. Thirdly, only a small flexible suction can be passed through the instrument channel to accomplish removal of debris and blood. This can be done only intermittently since the channel is also used for other instrument used in surgery. This makes it inefficient and increases the duration of surgery and is time consuming for the surgeon. Further since the suction is flexible it cannot provide enough counter pressure to aid in tissue dissection.

Endoscopic brain surgery in the second way has problems as well. First, a large part of the space in the port is occupied by the endoscope. Using two instruments through the rest of the narrow space in the port to accomplish dissection at a depth is difficult and tedious. It results in scissoring of instruments. Second, since the endoscope is fixed to a holder the surgeon has to constantly stop and reposition the endoscope to visualize the area of interest. This increases the duration of surgery, tires the surgeon and increases the risk of the operation.

Finally, in prior art, the endoscope accumulates debris and blood on the lens from the splatter. It has to be repeatedly removed from the field of surgery and cleaned. This is time consuming and frustrating for the surgeon.

Komiya U.S. Pat. No. 4,043,323 described a medical instrument attached to an endoscope. However, this device does not provide any means of suction or irrigation.

Heckele U.S. Pat. No. 4,881,523 describes an endoscope for endonasal surgery comprising a suction and flushing shaft. The suction is contained within the shaft and does not protrude beyond and hence cannot be used for bimanual dissection.

Arias U.S. Pat. No. 5,429,596 describe a similar device in which an electrocautery can be inserted through a side channel.

McFarlin U.S. Pat. No. 5,667,478 discloses a fiberoptic scope that can be attached to a medical instrument. Such does not provide a means for irrigation or suction. Further a fiberoptic scope has poor image resolution.

Iafrati U.S. Pat. No. 6,129,661 describes an endoscopic instrumentation with a working channel for use in subfascial endoscopic surgery. This instrumentation cannot be used for bimanual dissection.

Rudischhauser U.S. Pat. No. 6,471,639 discloses a rigid shaft with multiple channels for the endoscope, instrumentation, light and irrigation or suction. The suction channel is contained within the shaft and does not protrude beyond the shaft limiting its usefulness in bimanual dissection

Kent U.S. Pat. No. 6,585,642 discloses a flexible endoscope with a removable suction tube. Such flexible endoscopes cannot be used for bimanual dissection instrumentation.

Sood US Patent Application 20120265008 disclosed an endoscope shaft with an irrigation channel and a suction channel at 6 O'Clock position to which different suction tips of desired length could be attached for bimanual dissection. However, like prior art the position of the suction tips in relation to the endoscope could not be varied.

Van Lindert E J and Grotenhuis J Andre (New Endoscope Shaft for Endoscopic Transphenoidal Pituitary Surgery. Operative Neurosurgery. Vol 57 July 2005 ONS 203-205) describe a shaft through which a flexible suction catheter could be placed at a desired depth for suction and irrigation, but the flexible catheter does not offer the stiffness required to dissect tissues. The position of the catheter in relation to the endoscope is fixed at 6 o'clock.

Cutler A R, Kaloostian S W, Ishiyama A, Frazee J G: Two-handed endoscopic-directed vestibular nerve sectioning: case series and review of the literature. J Neurosurg 117:507-513, 2012 Describe use of Frazee II neurosendoscope (KARL STORZ GmbH & Co. KG Mittelstr. 8, 78532 Tuttlingen Postfach 230, 78503 Tuttlingen Deutschland) which has an endoscope shaft with an attached rigid suction whose depth can be varied, however like prior art, its position in relation to the endoscope is fixed at 6 o'clock.

It is the intent of the present invention to allow use of endoscope and yet permit two handed tissue dissection, ability to constantly suction debris and blood from the area of interest and permit surgery without need for constantly releasing the endoscope from the holder to reposition it to the area of interest while retaining the advantages of endoscopic surgery that is, small incision, better illumination and higher magnification.

### BRIEF SUMMARY OF THE INVENTION

The primary object of the invention is to be able to do two handed dissection of tissues during endoscopic brain surgery.

Another object of the invention is to be able to position, the suction in any position in relation to the endoscope.

Another object of the invention is to be able to adjust the length of the suction in relation to the endoscope.

Another object of the invention is to be able to control the strength of the surgical suction

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a handle for endoscope for two handed surgical dissections during endoscopic brain surgery comprising: A Rigid surgical suction whose position in relation to the endoscope can be varied from 0 to 360 degrees, Channel for endoscope, Channel for irrigation adjacent to the surgical suction, Suction control hole on the handle.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1A shows the prior art of brain surgery with a surgical microscope

FIG. 1B shows prior art of brain surgery with an endoscope

FIG. 1C show prior art of brain surgery using a port with an endoscope fixed in position

FIG. 2A shows the exploded view of the present invention

FIG. 2B shows the sectional view the invention

FIG. 3 shows the perspective view of the present invention

FIG. 4 shows the modality of use of the present invention with a port

FIG. 5 shows the use of present invention without a port

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

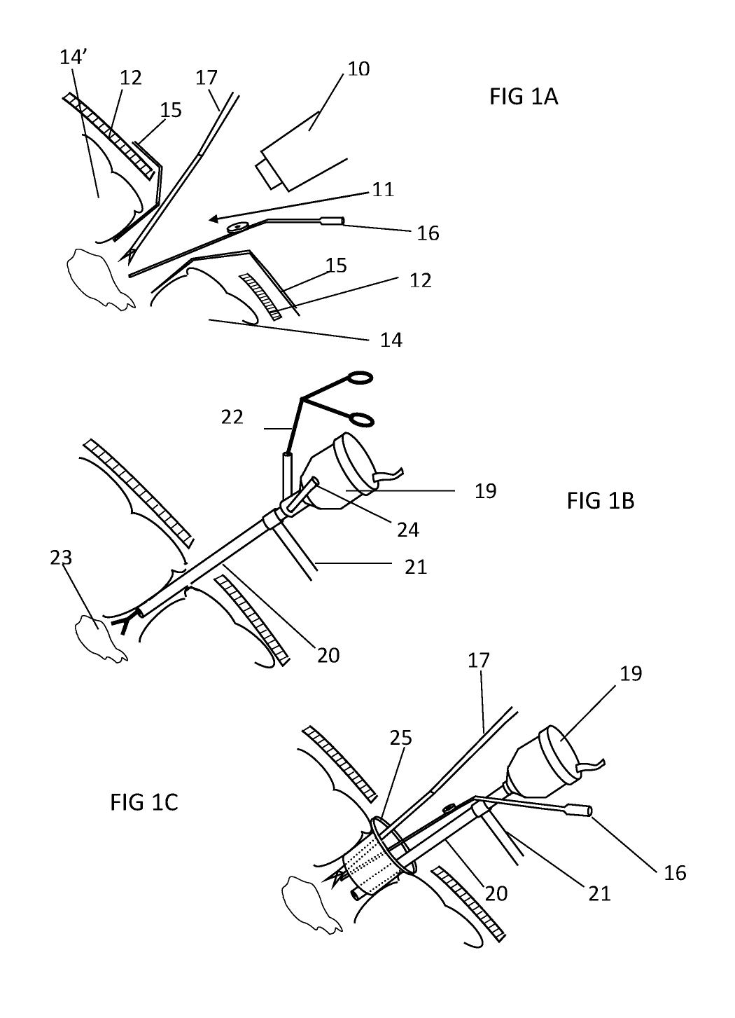

FIG. 1A shows the prior art of brain surgery with a microscope 10. An opening, 11 in the bone 12 allows tissue 14 & 14′ to be spread apart with a retractor blade 15. The surgeon operates holding two instruments 16, generally a surgical suction, and 17, generally some forceps, dissecting instrument, scissor or any of the myriad instruments suited for the procedure being performed. The surgical suction keeps the field clear of blood and debris and in conjunction with the second instrument is used for dissecting tissue planes in a two handed bimanual fashion.

FIG. 1B shows prior art of brain surgery with an endoscope 19, that passes through a shaft 20 at 6 o'clock position. It may be fixed to a holder 21. The coaxial instrument channel, generally at 12 o'clock position, is used to push an instrument 22 with jaws or scissors into the area of interest and tissue 23 that is to be removed or repaired. Bi-manual dissection of the tissue is difficult since there is a single instrument that can move in only one plane, that is, in and out. Some endoscopes may have additional co-axial channel 24 for using a flexible suction or irrigation to remove debris and blood.

FIG. 1C show prior art of brain surgery using a port 25 placed through the brain tissue to the area of interest. The endoscope 19 is fixed to a holder 21 at the shaft 20. Two instruments 16 & 17 are then used through the limited space in the port to accomplish surgery. The shaft 20 is released from the holder 21 and re-positioned every time the area of interest changes, to obtain a better view of the operative field. Endoscope 19 may need to be removed from the shaft periodically and cleaned if the lens accumulates debris or blood and obscures vision.

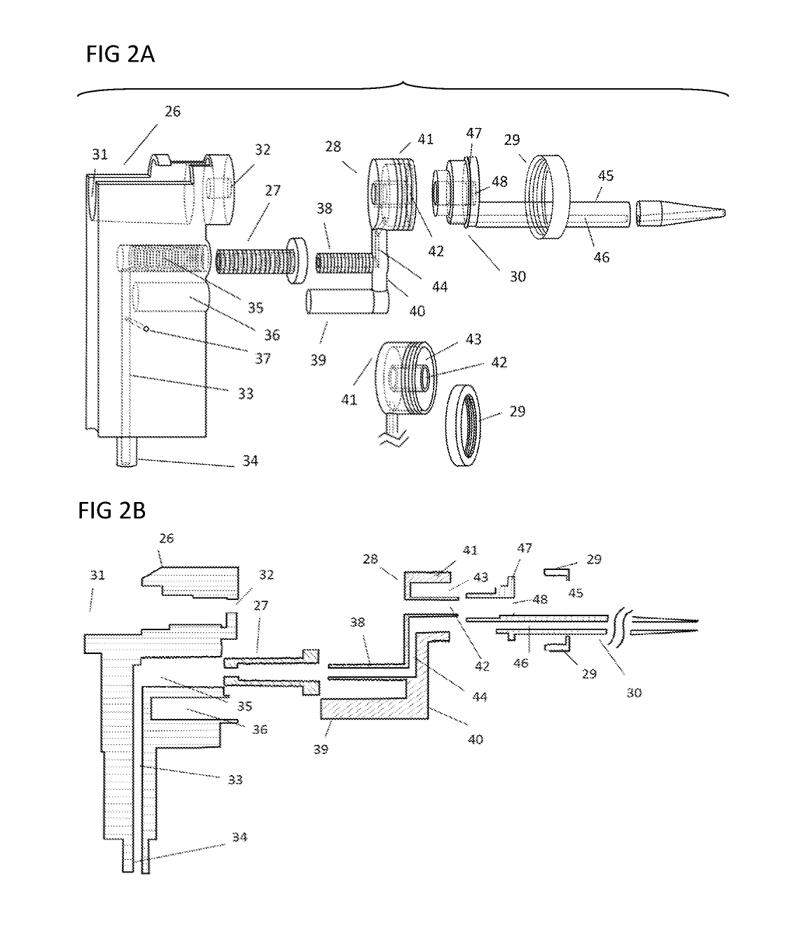

FIG. 2A is the exploded view and FIG. 2B sectional view that shows the present invention comprising a handle piece 26, control screw 27, suction piece 28, nut 29 and suction 30. The handle piece 26 has slot 31 for the endoscope body and hole 32 for the endoscope shaft. Suction channel 33 runs in the handle piece from the connector 34 to cylindrical receptacle 35 which has threads in clockwise direction. Cylindrical receptacle 36 below 35 is not threaded. Control hole 37 connects to the channel 33.

Control screw 27 is hollow with clockwise threads on the outside and anticlockwise directions on the inside. It fits into the cylindrical receptacle 35 of the handle piece 26 with corresponding clockwise threads.

Suction piece 28 has an arm 38 with threads in anticlockwise direction that fits into the inside of the hollow of the control screw 27. Second arm 39 fits into the cylindrical receptacle 36 of the handle piece 26 and is of the same length or longer than the arm 38. The arms 38 and 39 connect to the head 41 through a tube 40. The head 41 has a central channel 42 for the endoscope shaft and space 43 for the suction 30. The head 41 has threads of the outside that correspond to the threads on the inside of the nut 29. A channel 44 runs in the arm 38 through the tube 40 to connect to the space 43 in the head 41.

Suction 30 has a suction tube 45 with a channel 46 running through and through. And one end of the suction tube 45 is connected to the head 47 with channel 48 that fits around the central channel 42 of the head 41 of the suction piece 28. When head 47 is placed in the space 43 and nut 29 tightened, the surface 49 of the head seals against the inside of the head 41.

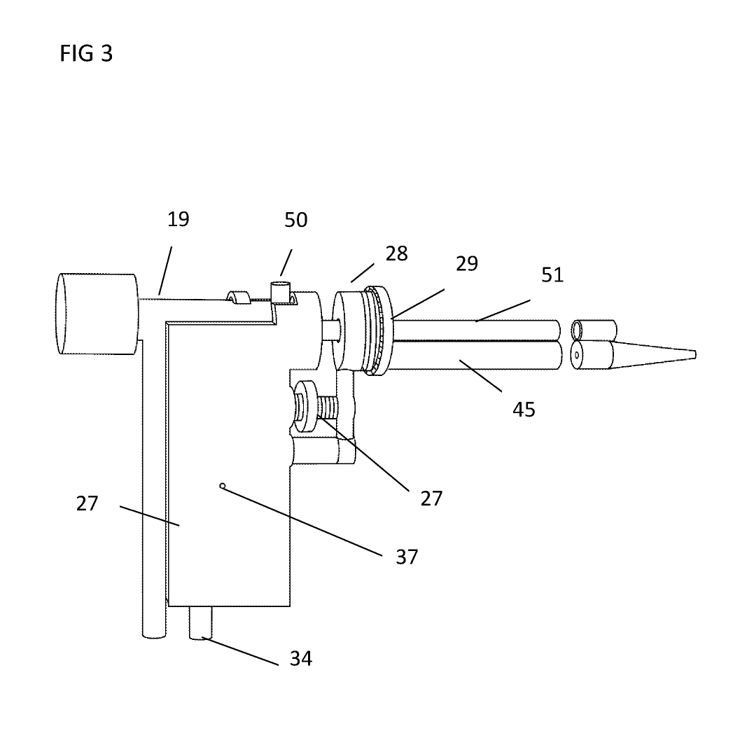

FIG. 3 show the perspective view of the assembled device with the endoscope 19 that fits in the slot 31 of the handle piece, irrigation can be done through the port 50 of the endoscope. The control screw 27 can be used to move the suction piece 28 and the attached suction 30 with the suction tube 45 forwards or backwards. By loosening the nut 29, the position of the suction in relation to the endoscope shaft 51 and be adjusted to any position between 0-360 degrees and then the nut can be tightened to secure it in that position. Suction pressure can be applied to the connector 34 and the amount of suction can be controlled by closing control hole 37 with the thumb or finger of the hand holding the handle piece 26.

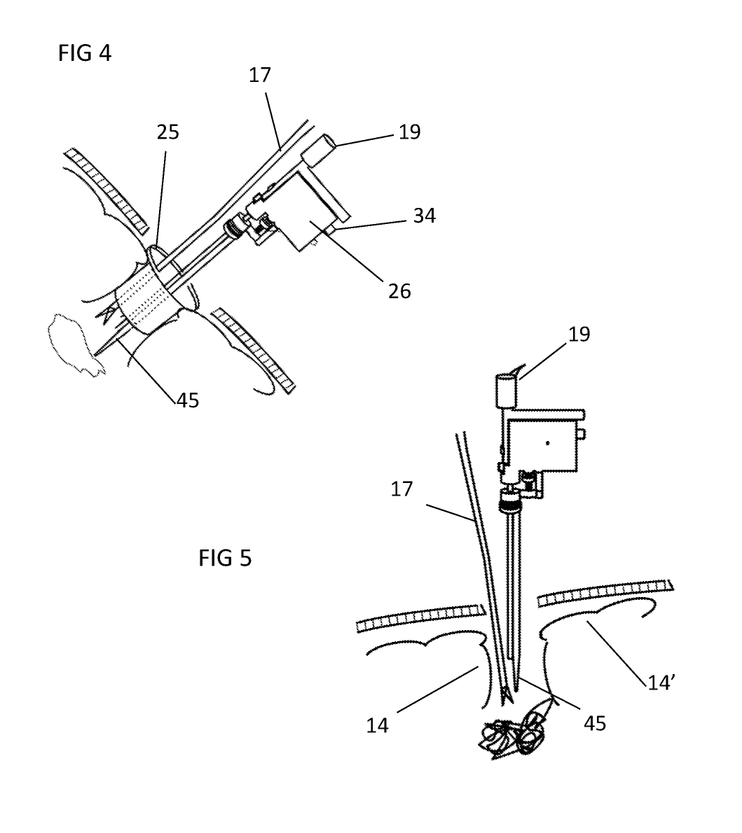

FIG. 4 shows the modalities of use of the present invention. The device is introduced through a port 25 and is held in the left hand. A second instrument 17 is used through the port in the right hand. Under visualization provided by the endoscope 19, the suction tube 45 is used in conjunction with instrument 17 to do two handed bimanual dissection of the tissues. The suction tube 45 constantly keeps the field of surgery clear of blood and debris and it strength is modulated by the degree to which the control hole 37 is covered by the thumb of the hand holding the handle piece 26. Irrigation fluid may be run continuously or intermittently through the port 50 and suction applied at connector 34. As the surgeon moves the suction to the field of interest by moving the handle piece 26 held by the left hand, the endoscope by virtue of being in the handle piece is also directed toward that area of interest obviating the need for constant releasing it from the holder and repositioning it as in the prior art.

FIG. 5 show the alternate way of using the device without a port. The device is held in the left hand and slipped under the surface of the brain or between the two hemispheres of the brain 14 and 14′. A second instrument in 17 held in the right hand is then able to accomplish surgery in conjunction with suction tube 45 in the device in a two handed bimanual fashion.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A handle for endoscopic micro-neurosurgery comprising:
Channel for endoscope;
Rigid suction;
Surgical suction control hole on the handle;

2. A handle for endoscopic micro-neurosurgery in claim 1, which has a rigid surgical suction that is longer than the shaft

3. A handle for endoscopic micro-neurosurgery in claim 1, which has a rigid surgical suctions tube that can be adjusted around the endoscope from 0-360 degrees.

4. A handle for endoscopic micro-neurosurgery in claim 1, which has a surgical suction tip can be adjusted in relation to the tip of the endoscope so that it can be brought closer to the tip to the endoscope or taken further away from it.
